(19) United States
(12) Patent Application Publication
(10) Pub. No.: US 2026/0108362 A1
(43) Pub. Date: Apr. 23, 2026

(54) INTERBODY DEVICE, SYSTEM, AND METHOD, FOR MINIMALLY INVASIVE KAMBIN LUMBAR LATERAL INTERBODY FUSION

(71) Applicant: AA Technologies, LLC, Toledo, OH (US)

(72) Inventors: Anand Agarwal, Ottawa Hills, OH (US); Niraj Vasavada, Ahmedabad (IN); Darshak Shah, Ahmedabad (IN)

(21) Appl. No.: 19/350,506

(22) Filed: Oct. 6, 2025

Related U.S. Application Data
(60) Provisional application No. 63/703,343, filed on Oct. 4, 2024.

(51) Int. Cl. A61F 2/44; A61F 2/30; A61F 2/46

(52) U.S. Cl. CPC A61F 2/447; A61F 2/30771; A61F 2/4611; A61F 2002/3008; A61F 2002/30217; A61F 2002/30224; A61F 2002/30326; A61F 2002/30784; A61F 2002/30904; A61F 2002/3093; A61F 2002/4629; A61F 2002/4677

(57) ABSTRACT

An interbody device for a surgical procedure having an outer body made of a radiopaque material and an insertion portion having a first width at a first end of the insertion portion that is less than a second width at a second end of the insertion portion. The insertion portion configured for entering into a disc space and the interbody device including a top surface having a convex shape.

### CROSS-REFERENCE TO RELATED APPLICATIONS

This application claims the benefit of U.S. Provisional Application No. 63/703,343, filed on Oct. 4, 2024. The entire disclosure of the above application is incorporated herein by reference.

### FIELD

The present technology relates to spinal fusion devices and procedures and, more particularly, to minimally invasive interbody devices and associated procedures for lateral lumbar interbody fusion through Kambin's triangle.

### INTRODUCTION

This section provides background information related to the present disclosure which is not necessarily prior art.

Spinal fusion is a surgical procedure used to correct problems with the vertebrae of the spine. The procedure is designed to minimize motion between vertebral segments that may cause pain and to provide stability to the spine. Certain spinal fusion techniques involve invasive surgery requiring one or more large incisions, significant muscle dissection, and extended recovery times. There is a trend towards minimally invasive spinal fusion surgical procedures designed to reduce tissue damage and blood loss, as well as minimize recovery time compared to more invasive surgeries. One area of focus includes the development of minimally invasive procedures with a focus on lateral approaches to the lumbar spine.

Lateral lumbar interbody fusion (LLIF) is one minimally invasive technique that approaches the spine from the side rather than from the back or front. This approach may reduce injury to back muscles, bones, and ligaments. However, LLIF techniques still require dissection through the psoas muscle, which may cause postoperative pain and potential nerve injury.

In contemplating approaches to spinal surgery, the Kambin's triangle is an anatomical space though which an intervertebral disc may be accessed from a posterolateral approach. Kambin's triangle is bordered by an exiting nerve root laterally, a traversing nerve root medially, and a superior endplate of an inferior vertebral body inferiorly. Utilizing Kambin's triangle for disc access may reduce a risk of nerve injury compared with other approaches.

Interbody devices for spinal fusion, or cages, include implants placed between vertebrae to maintain disc height and promote fusion. Certain interbody fusion devices may be made entirely of titanium or polyetheretherketone (PEEK). While titanium offers excellent strength and osseointegration properties, the material is radiopaque. As a result, radiographic assessment of spinal fusion including subsequent bone growth can be challenging due to titanium's radiopacity. PEEK, on the other hand, is radiolucent but may be suboptimal with respect to bone growth compared with titanium.

Interbody devices and insertion techniques for far lateral approaches through Kambin's triangle may present certain limitations. For example, interbody devices may include a bullet-shaped design that requires forceful insertion. This may cause damage to vertebral endplates. Additionally, existing systems for inserting and placing interbody devices often utilize radiopaque components, making it difficult to visualize the interbody device during the procedure.

There is a continuing need for an improved interbody device, system, and method, for minimally invasive lateral lumbar interbody fusion through Kambin's triangle. Desirably, the interbody device would be configured for easier and safer insertion, promote lordosis restoration, facilitate radiographic assessment of fusion, and minimize the risk of endplate damage and nerve injury during placement.

### SUMMARY

In concordance with the instant disclosure, an improved interbody device, system, and method, for minimally invasive lateral lumbar interbody fusion through Kambin's triangle, is configured for easier and safer insertion, promotes better lordosis restoration, facilitates radiographic assessment of fusion, and minimizes the risk of endplate damage and nerve injury during placement, has surprisingly been discovered. The present technology includes articles of manufacture, systems, and processes that relate to minimally invasive interbody devices for lateral lumbar interbody fusion through Kambin's triangle, also known herein as “Kambin Lumbar Lateral Interbody Fusion” or KLIF.

In certain embodiments, an interbody device for a surgical procedure may include an outer body made of a radiopaque material. The interbody device may further include an insertion portion having a first width at a first end of the insertion portion that is less than a second width at a second end of the insertion portion. The insertion portion may be configured for entering into a disc space. A top surface of the interbody device may have a convex shape.

In certain embodiments, a system for a surgical procedure may include an interbody device. The interbody device may include an outer body made of a radiopaque material. The interbody device may further include an insertion portion having a first width at a first end of the insertion portion that is less than a second width at a second end of the insertion portion. The insertion portion may be configured for entering into a disc space. A top surface of the interbody device may have a convex shape. The system may further include a working cannula, wherein the working cannula includes a radiolucent portion.

In certain embodiments, a method of performing a procedure may include a first step of providing an interbody device. The interbody device may include an outer body made of a radiopaque material. The interbody device may further include an insertion portion having a first width at a first end of the insertion portion that is less than a second width at a second end of the insertion portion. The insertion portion may be configured for entering into a disc space. A top surface of the interbody device may have a convex shape. A surgical pathway to the disc space may be created through Kambin's triangle. The interbody device may be inserted through the surgical pathway and into the disc space using an insertion instrument removably coupled to the interbody device. The insertion instrument may be decoupled from the interbody device. Fusion may be allowed to occur between adjacent vertebrae.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1 is a block diagram illustrating an interbody device, according to certain embodiments of the present disclosure;

FIG. 2 is a block diagram illustrating a system including an interbody device, according to certain embodiments of the present disclosure;

FIG. 3 is a flowchart illustrating a method of performing a procedure using an interbody device, according to certain embodiments of the present disclosure;

FIG. 4 is a front perspective view of an interbody device, according to certain embodiments of the present disclosure;

FIG. 5 is a rear perspective view of the interbody device shown in FIG. 4;

FIG. 6 is a side elevational view thereof;

FIG. 7 is a cross-sectional view thereof;

FIG. 8 is a front perspective view of another interbody device, according to certain embodiments of the present disclosure;

FIG. 9 is a rear perspective view of the another interbody device shown in FIG. 8;

FIG. 10 is an exploded view thereof; and

FIG. 11 is a perspective view of a system including an interbody device being positioned relative to upper and lower vertebrae, according to certain embodiments of the present disclosure.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement may allow some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The present technology improves surgical procedures such as a minimally invasive lateral lumbar interbody fusion procedure using an interbody device configured for insertion through Kambin's triangle, a three-dimensional anatomical right triangle in the lumbar spine. Kambin's triangle is defined by a base formed by a superior endplate of an inferior vertebral body, a height formed by a superior articulating facet, and a hypotenuse formed by an exiting superior nerve root. The interbody device may be configured for insertion at a far lateral side of a disc space through Kambin's triangle and may militate against tissue damage while improving a disc space height between an adjacent upper vertebra and lower vertebra, as well as post-operative outcomes. Advantageously, procedures using the interbody device may utilize a working cannula having a radiolucent portion and a cannulated, over the guidewire delivery method for enhanced precision.

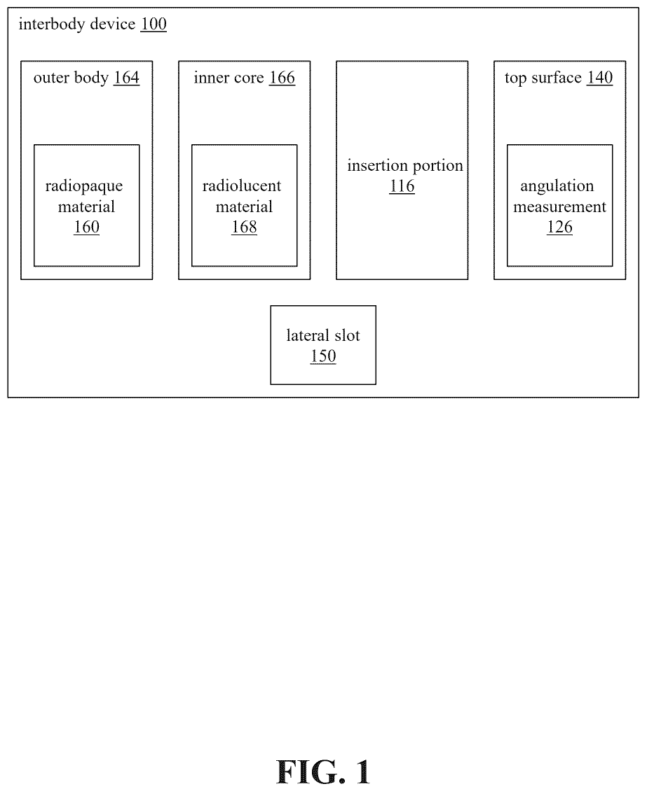

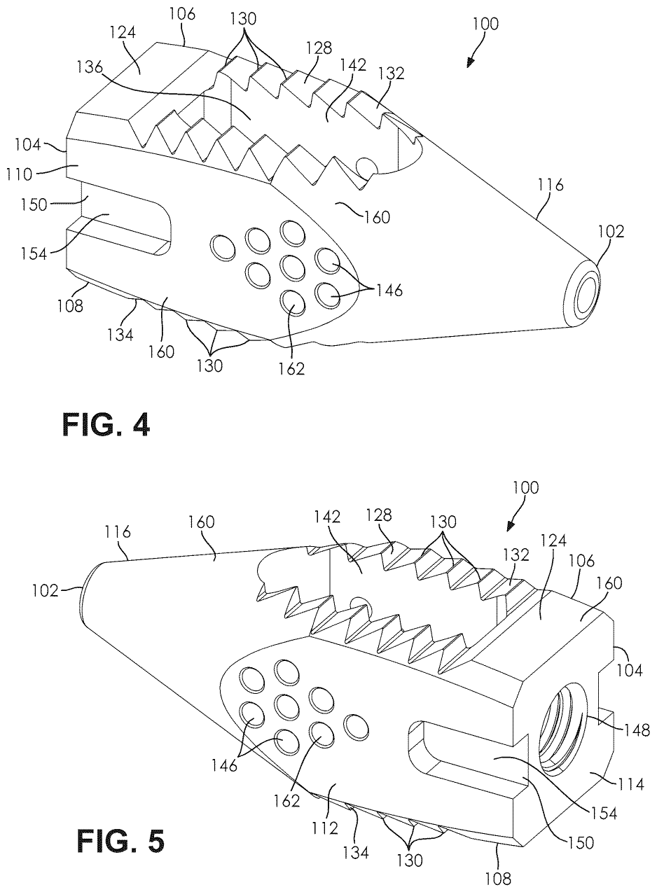

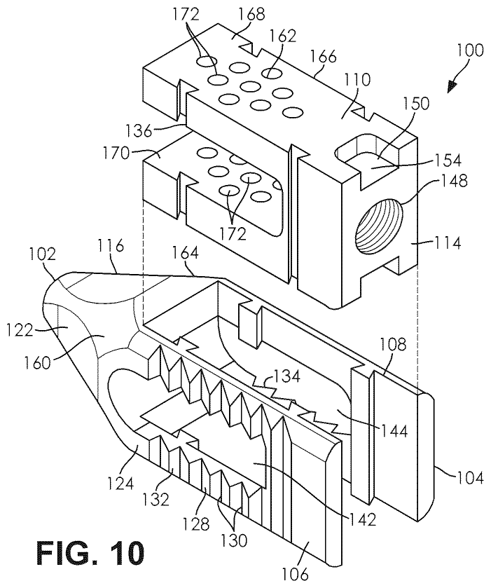

With reference to FIGS. 4-10, an interbody device 100 may have a first end 102 configured for insertion into a disc space 123, a second end 104 disposed opposite the first end 102, a top wall 106, a bottom wall 108, a first side wall 110, a second side wall 112, and a rear wall 114. The interbody device 100 may be fabricated using any suitable strong, biocompatible material or combination of materials, as determined by one of skill in the art. As examples, the interbody device 100 may include a metal such as titanium, a plastic such as carbon fiber polyetheretherketone (PEEK), a transparent PEEK, a hydroxyapatite (HA), a ceramic material, a synthetic material, a human allograph bone such as a human cortical graft, or any combination thereof. The interbody device 100 may be any suitable size and may have any desired dimensions. It should be appreciated that the interbody device 100 may be manufactured in a range of sizes and dimensions. Substrative manufacturing, additive manufacturing, and/or any other suitable means for manufacturing the interbody device 100 may be employed, as determined by a skilled artisan. The interbody device 100 may be a single integral component, as shown in FIGS. 4-7, or may be a plurality of components integrally, permanently, semi-permanently, or removably coupled to one another, as shown in FIGS. 1 and 8-10.

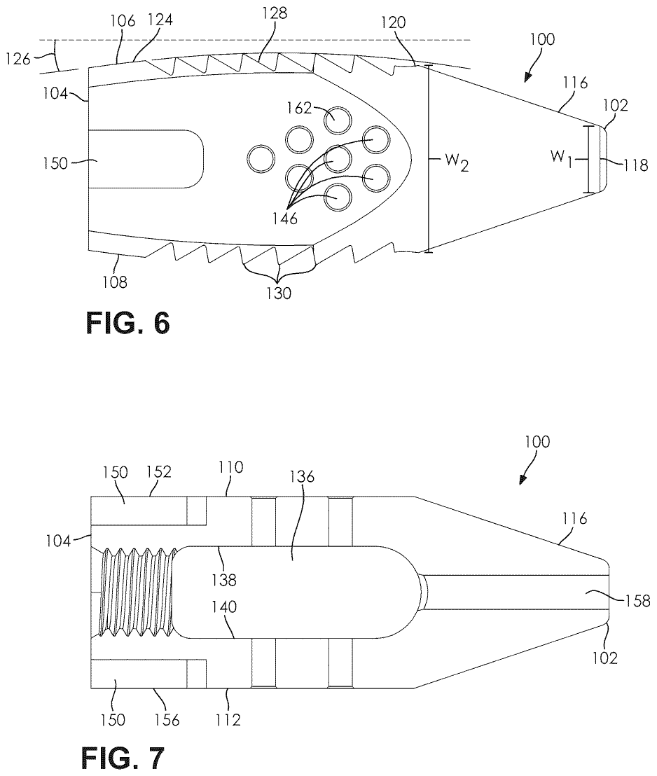

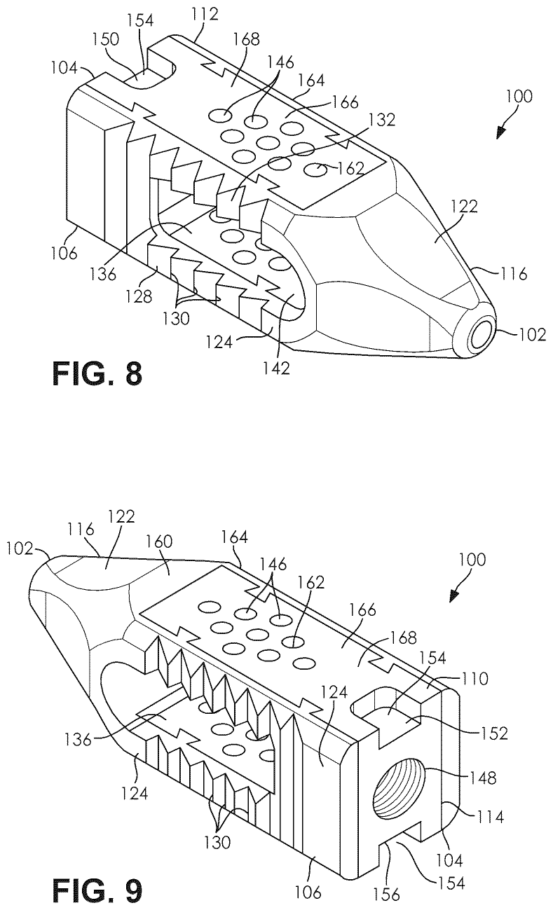

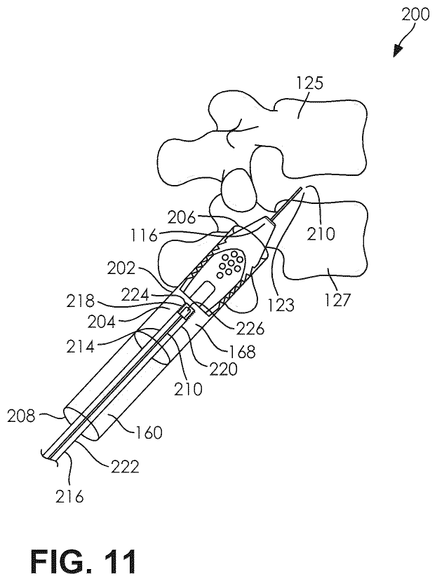

The interbody device 100 may include an insertion portion 116 adjacent the first end 102 of the interbody device 100. The insertion portion 116 may have an insertion portion first end 118 adjacent the first end 102 of the interbody device 100 and an insertion portion second end 120 disposed opposite the insertion portion first end 118, as shown in FIG. 6. A first width W1 of the insertion portion first end 118 may be less than a second width W2 of the insertion portion second end 120. In certain embodiments, the width of the insertion portion 116 may gradually increase from the insertion portion first end 118 to the insertion portion second end 120. As shown in FIGS. 4-7 the insertion portion 116 may have a substantially cone-like shape. In certain embodiments, as shown in FIGS. 8-10, the insertion portion 116 may be substantially cylindrical in shape at the insertion portion first end 118 and may have a concave side wall surface 122 extending from the insertion portion first end 118 to the insertion portion second end 120. In certain embodiments, the insertion portion 116 may have a shape that is substantially similar to that of a dolphin beak or rostrum. Advantageously, the insertion portion 116 may be configured to facilitate easy insertion into the disc space 123 between an upper vertebra 125 and a lower vertebra 127, as shown in FIG. 11, to thereby militate against vertebral endplate and nerve damage during a procedure.

As shown in FIG. 6 the top wall 106 of the interbody device 100 may have a top surface 124 having a convex shape extending from the first end 102 of the interbody device 100 and/or the insertion portion second end 120 to the second end 104 of the interbody device 100. The convex shape of the top surface 124 of the interbody device 100 may have an angulation measurement 126 from greater than 0 degrees to 30 degrees. In certain embodiments, the convex shape of the top surface 124 of the interbody device 100 may have an angulation measurement 126 from greater than 0 degrees to 20 degrees. It should be appreciated that any desired angulation measurement 126 may be employed. Advantageously, the convex shape of the top surface 124 may improve spinal curvature and militate against lordosis by positioning the upper vertebra 125 to tilt backwards. The interbody device 100 may be manufactured in a variety of sizes, dimensions, and angulation measurements 126, as examples, such that a user may select a desired interbody device 100 to achieve an optimal fit between the upper vertebra 125 and the lower vertebra 127, as well as a desired degree of lordosis correction during a procedure. The convex shape of the top surface 124 of the interbody device 100 may be further configured to allow the interbody device 100 to more atraumatically slide past sensitive components in and around a spine such as a nerve root, as an example.

The interbody device 100 may include a serrated surface 128 having a plurality of teeth 130 or ridges extending outwardly therefrom. In certain embodiments, the top wall 106 of the interbody device 100 may include a first serrated surface 132 and the bottom wall 108 may include a second serrated surface 134, as shown in FIG. 5. The serrated surface 128 may be configured to improve stability during implantation of the interbody device 100 and militate against dislodging and/or migration of the interbody device 100 post procedure.

With reference to FIGS. 4-5 and 7, a cavity 136 may be formed centrally between the top wall 106, the bottom wall 108, the first side wall 110, and the second side wall 112. In certain embodiments, the cavity 136 may be defined by a first inferior surface 138 of the first side wall 110, a second inferior surface 140 of the second side wall 112, a first opening 142 disposed in the top wall 106, and a second opening 144 disposed in the bottom wall 108 of the interbody device 100. The cavity 136 may be configured to receive material such a bone graft material during a bone graft, as an example. The first opening 142 and the second opening 144 may be configured to promote fusion and bone growth.

The interbody device 100 may include an aperture 146 or a plurality of apertures 146 disposed in one or both of the first side wall 110 and the second side wall 112. In certain embodiments, the plurality of apertures 146 may be arranged in a honeycomb formation, as shown in FIGS. 4-6 and 8-10. Advantageously, each aperture 146 may allow a user to visualize a bone graft and subsequent bone formation using radiographic imaging. Additionally, the aperture 146 may enhance integration of the interbody device 100 and bone ingrowth.

An aperture, such as a threaded hole 148, may be disposed centrally in the rear wall 114 of the interbody device 100 and may be configured to removably couple to a surgical instrument, as shown in FIGS. 5, 7, 9, and 10. The threaded hole 148 may be further configured to couple to a bone graft delivery system and receive a bone graft material during a procedure. It should be appreciated that the aperture (e.g., threaded hole 148) may be any suitable shape, size, and configuration capable of coupling to standardized and/or other surgical instruments, as determined by one of skill in the art. The threaded hole 148 may be configured to decouple from the surgical instrument using any suitable releasing mechanism, such as a controlled rotational releasing mechanism, as an example.

With renewed reference to FIGS. 4-10, the interbody device 100 may include a lateral slot 150 configured to removably couple to a surgical instrument. The lateral slot 150 may militate against undesirable rotation and movement of the interbody device 100 during placement of the interbody device 100 between the upper vertebra 125 and the lower vertebra 127. The lateral slot 150 may be configured to engage the surgical instrument using any suitable mating and/or locking mechanism, such as a sliding or insertion mechanism, as determined by a skilled artisan. The lateral slot 150 may disengage from the surgical instrument once the interbody device 100 is desirably positioned and secured between the upper vertebra 125 and the lower vertebra 127 using any suitable controlled releasing mechanism.

As shown in FIGS. 4, 5, and 7, a first lateral slot 152 may be defined by a recessed portion 154 disposed in one or both of the first side wall 110 and the rear wall 114 of the interbody device 100, and a second lateral slot 156 may be defined by a recessed portion 154 disposed in one or both of the second side wall 112 and the rear wall 114 of the interbody device 100, according to certain embodiments. It should be appreciated that the lateral slot 150 may include any groove, recess, opening, or other structural component configured to removably couple to a surgical instrument and militate against undesirable rotation and movement of the interbody device 100 during insertion and placement of the interbody device 100.

The interbody device 100 may be an expandable and/or adjustable interbody device 100, thereby allowing for in situ expansion and/or adjustment of the interbody device 100 during a procedure. As examples, a height, a width, and/or an angulation measurement 126 may be adjusted prior to or during a procedure. Any suitable adjustment mechanism may by employed such as a threaded expansion or a hydraulic system, as examples. Advantageously, expansion and/or adjustment of the interbody device 100 may allow the user to adjust the interbody device 100 based on a size and/or anatomy of a patient undergoing a procedure, thereby optimizing the fit and positioning of the interbody device 100 in the disc space 123 between the upper vertebra 125 and the lower vertebra 127, as well as the desired degree of lordosis correction.

The interbody device 100, as shown in FIG. 7, may include a cannulation channel 158 and may be configured for guided insertion over a guidewire extending through the cannulation channel 158. Advantageously, cannulated, over-the-guidewire delivery of the interbody device 100 may optimize precise placement of the interbody device 100 by allowing the user to use the guidewire as a reference during the insertion process. Additionally, the cannulated, guided insertion may facilitate minimally invasive delivery and placement of the interbody device 100, thereby militating against tissue damage and improving patient outcomes such as recovery time.

In certain embodiments, as shown in FIGS. 4 and 5, the interbody device 100 may be a single, unitary component made of any suitable radiopaque material 160, such as titanium, as an example. The radiopaque material 160 may optimize visibility of the interbody device 100 during and after placement of the interbody device 100, optimize structural support including compressive strength and dimensional stability, and enhance osseointegration. One or more of the first opening 142 disposed in the top wall 106, the second opening 144 disposed in the bottom wall 108, and the aperture 146 may be configured to provide a radiolucent window 162 for visualization during and after a procedure for visualizing bone material during and/or after a bone graft. It should be appreciated that one or more of first opening 142 disposed in the top wall 106, the second opening 144 disposed in the bottom wall 108, and the aperture 146 may also be configured to promote fusion and bone growth post procedure.

In certain embodiments, the interbody device 100, may include a plurality of components removably, permanently, or semi-permanently coupled together as shown in FIGS. 1 and 8-10. The interbody device 100 may have an outer body 164 fabricated from a radiopaque material 160 such as titanium, as an example. The outer body 164 may optimize visibility of the interbody device 100 during and after placement of the interbody device 100 using radiographic imaging, optimize structural support including compressive strength and dimensional stability, and promote osseointegration with an upper vertebra and a lower vertebra. The interbody device 100 may include an inner core 166 fabricated using a radiolucent material 168, such as PEEK, as an example. The radiolucent inner core 166 may allow for improved visualization of the bone graft during a procedure and the fusion process including bone growth in post-operative radiographs, while maintaining the structural integrity of the interbody device 100. The inner core 166 may include an inner core opening 170 and/or an inner core aperture 172 in one or more of the top wall 106, the bottom wall 108, the first side wall 110, and the second side wall 112 configured to promote fusion and bone growth during and after a bone graft. The inner core 166 may include, form, abut, and/or be disposed adjacent to one or more of the top wall 106, the bottom wall 108, the first side wall 110, the second side wall 112, and the rear wall 114, according to certain embodiments.

It should be appreciated that one or more components of the interbody device 100 may be manufactured using any suitable mechanism or combination of mechanisms. One of ordinary skill in the art may select any suitable materials or combination of materials, as desired. The interbody device 100 may be one unitary component or a combination of multiple components, as determined by one of skill in the art. The interbody device 100 may be configured for use during a surgical procedure, such as a tubular or endoscopic surgical procedure, as examples.

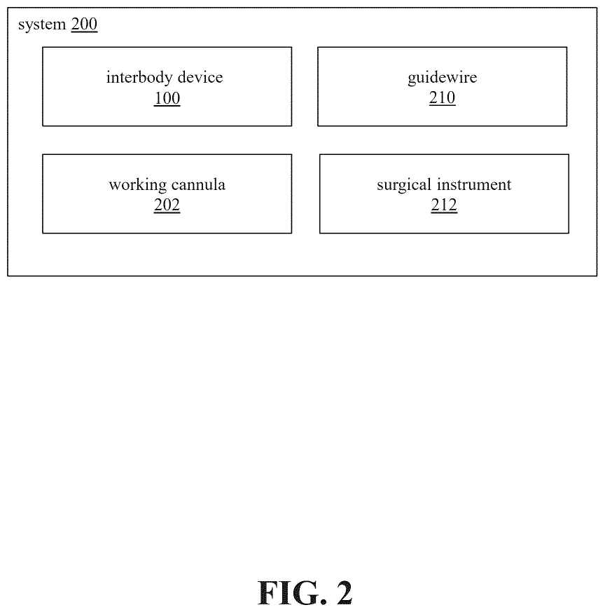

As shown in FIGS. 2 and 11, a system 200 used for procedures such as a minimally invasive lateral lumbar interbody fusion procedure may include the interbody device 100 and a working cannula 202. The working cannula 202 may be fabricated using any suitable flexible, durable, biocompatible material or combination of materials. In certain embodiments, a radiolucent portion 204 of the working cannula 202 may be fabricated using a radiolucent material 168, thereby allowing for real-time visualization of the interbody device 100 during insertion and/or placement of the interbody device 100 in the disc space 123 between the upper vertebra 125 and the lower vertebra 127 using radiographic imaging. The radiolucent portion 204 may be fabricated using any suitable material or combination of materials such as PEEK or carbon fiber reinforced PEEK. It should be appreciated that any suitable radiolucent material 168 or combination of materials may be used to form the radiolucent portion 204 of the working cannula 202, as desired.

In certain embodiments, as shown in FIG. 11, the working cannula 202 may have a first end 206 configured for placement at or adjacent the disc space 123. The first end 206 of the working cannula 202 may be fabricated using a radiolucent material 168 and a second end 208 disposed opposite the first end 206 may be fabricated using a radiopaque material 160 such as stainless steel, as an example. It should be appreciated that any desired length of the working cannula 202 may be fabricated using radiolucent material 168 or materials and any desired length of the working cannula 202 may be fabricated using radiopaque material 160 or materials. As an example, approximately one third of the working cannula 202 may be the radiolucent portion 204 fabricated using radiolucent material 168 at the first end 206. In certain embodiments, the working cannula 202 may be fabricated using only a radiolucent material 168 or materials. Advantageously, the radiolucent portion 204 of the working cannula 202 may allow the user to visualize the interbody device 100 during placement of the interbody device 100 in the disc space 123 between the upper vertebra 125 and the lower vertebra 127 using radiographic images. Accordingly, the radiolucent portion 204 may enhance placement and positioning of the interbody device 100 and militate against vertebral end plate and nerve damage.

The working cannula 202, according to certain embodiments, may include and/or be configured to slide over a series of cannulated dilators of increasing diameter (not shown). The series of cannulated dilators may be configured to be inserted sequentially, thereby gradually expanding tissue and creating a channel for insertion of the interbody device 100. The series of cannulated dilators may militate against tissue trauma and related surgical injuries during a procedure.

The system 200 may include a guidewire 210 over which the interbody device 100 may be guided. The guidewire 210 may be configured as a reference point for the user during a procedure and may enhance movement, insertion, and placement of the interbody device 100 during a minimally invasive procedure where direct visualization of the surgical pathway and/or the surgical site may be limited. The guidewire 210 may be fabricated using any flexible, strong, biocompatible material or combination of materials such as stainless steel, nitinol, tungsten, platinum, PTFE, and hydrophilic polymers, as examples. The guidewire 210 may be configured to guide the interbody device 100 along a predetermined path during a procedure.

The system 200 may include one or more surgical instruments 212 used in combination with the interbody device 100, the working cannula 202, and/or the guidewire 210 during a procedure. As examples, surgical instruments 212 used in combination with the interbody device 100, the working cannula 202, and/or the guidewire 210 may include an insertion instrument 214, one or more instruments configured for disc space preparation such as curettes and osteotomes (not shown), robotic instruments (not shown), and any other suitable surgical instruments 212, as determined by a skilled artisan. The surgical instruments 212 may be cannulated and configured to extend along a length of the guidewire 210, thereby optimizing controlled delivery of the surgical instruments 212, preparation of the disc space 123, the upper vertebra 125, and the lower vertebra 127, and insertion, placement, and positioning of the interbody device 100. The surgical instruments 212 may be configured to extend through the working cannula 202, thereby allowing the user to visualize the surgical instruments 212 during a procedure when the surgical instruments 212 are positioned adjacent the radiolucent portion 204 of the working cannula 202.

According to certain embodiments, the system 200 may include the insertion instrument 214, and the insertion instrument 214 may have an elongated body 216, a coupling mechanism 218 at a first end 220 of the insertion instrument 214 configured to removably couple to the interbody device 100, and a handle (not shown) at a second end 222 of the insertion instrument disposed opposite the first end 220. The elongated body 216 may be cannulated and configured for guided insertion over the guidewire 210, thereby enhancing surgical precision and militating against injury during a procedure. In certain embodiments, the coupling mechanism 218 may include a threaded portion 224 configured for engaging the threaded hole 148 of the interbody device 100 and at least one lateral flange 226 configured for insertion into the lateral slot 150 of the interbody device 100. Advantageously, the coupling mechanism 218 may allow for the insertion instrument 214 to securely couple to the interbody device 100 during insertion and placement of the interbody device 100 and easily decouple from the interbody device 100 once the interbody device 100 is placed a desired position. The coupling mechanism 218 may securely couple and easily decouple the insertion instrument 214 to and from the interbody device 100 using any suitable coupling mechanism and controlled release mechanism, as desired.

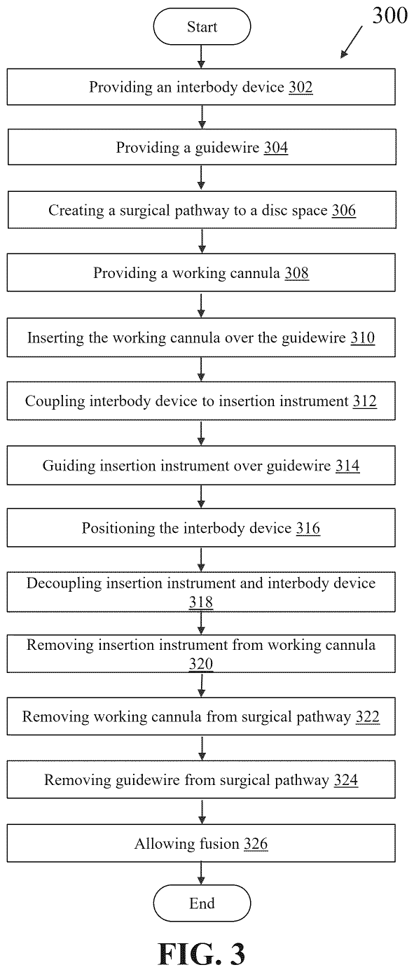

As shown in FIG. 3, a method 300 of performing a procedure such as a lateral lumbar interbody fusion procedure may include a first step 302 of providing the interbody device 100. A second step 304 of the method 300 may include providing the guidewire 210. Next, a third step 206 may include creating a surgical pathway to the disc space 123 through Kambin's triangle using the guidewire 210. In a fourth step 308, the working cannula 202 may be provided, and in a fifth step 310, the working cannula 202 may be inserted over the guidewire 210 and positioned in or adjacent the disc space 123. It should be appreciated that, in certain embodiments where the working cannula 202 is integrally formed with or removably coupled to a series of cannulated dilators, additional steps relating to insertion, set-up, and/or removal of the series of cannulated dilators, as examples, may be included in the method 300, as determined by one of skill in the art.

Following the creation of the surgical pathway and insertion of the guidewire 210 and working cannula 202, a step 312 may include coupling the interbody device 100 to the insertion instrument 214, and a step 314 may include guiding insertion instrument 214 including the interbody device 100 over the guidewire 210, through the working cannula 202, and along the surgical pathway into the disc space 123 using the insertion instrument 214. Additional steps relating to coupling the interbody device 100 to insertion instrument 214 such as steps relating to the coupling mechanism 218 and/or a locking mechanism may be included. A step 316 may include positioning the interbody device 100 between the upper vertebra 125 and the lower vertebra 127. It should be appreciated that any suitable additional steps relating to positioning and/or adjusting the interbody device 100, such as steps relating to expanding or otherwise adjusting the interbody device 100 to a desired size and configuration, as examples, may be employed in the method 300.

Once the interbody device 100 is in a desirable position, a step 318 may include decoupling the insertion instrument 214 from the interbody device 100. It should be appreciated that additional steps relating to decoupling the interbody device 100 from the insertion instrument 214 may include activating a decoupling mechanism and/or deactivating a locking mechanism, as examples.

In a step 320 of the method 300, the insertion instrument 214 may be removed from the working cannula 202. The working cannula 202 may be removed from the surgical pathway in a step 322, and the guidewire 210 may be removed from the surgical pathway in a step 324. Finally, in a step 326, fusion may occur between the upper vertebra 125 and the lower vertebra 127.

The method 300 may include additional steps relating to forming the surgical pathway such as creating an incision, as an example, selecting the interbody device 100, and preparing the disc space 123. As examples, additional steps may include cleaning the disc space 123 and preparing the disc space 123 using instruments such as curettes and osteotomes. Steps involving additional surgical instruments 212 such as the insertion, removal, and employment of additional surgical instruments 212 may also be included in the method 300. The method 300 may also include steps such as inserting a bone graft delivery system and/or inserting bone graft material into the interbody device 100. Steps including obtaining and observing radiographic images before, during, and after a procedure may also be included in the method 300. It should be appreciated that steps included in the method 300 may be performed in any suitable order, as determined by a skilled artisan. Steps may be repeated, omitted, and added, as desired.

The interbody device 100, system 200, and method 300 may advantageously enhance the safety and surgical outcomes associated with a procedure such as a minimally invasive lateral lumbar interbody fusion procedure. The configuration of the interbody device 100, including the insertion portion 116 and the convex top surface 124, may militate against injury, such as damage to a vertebral endplate and/or nerve, as examples, during insertion and positioning of the interbody device 100 in the disc space 123 between the upper vertebra 125 and the lower vertebra 127. The convex top surface 124 may improve lordosis and allow the interbody device 100 to gently slide past sensitive areas in and around the spine.

One or more of the cavity 136, the first opening 142, the second opening 144, the aperture 146, the inner core opening 170, and/or the inner core aperture 172 may additionally promote fusion and bone growth post-procedure. The cavity 136, the first opening 142, the second opening 144, the aperture 146, the inner core opening 170, and/or the inner core aperture 172 may also optimize visualization during and after a procedure by providing a radiolucent window 162 on radiographic images. In certain embodiments having an inner core 166 fabricated from a radiolucent material 168, visualization during and after a procedure may be further enhanced. More specifically, the radiolucent material 168 may allow visualization of bone graft material during a procedure and subsequent fusion and bone growth post procedure in radiographs.

Cannulated, over-the-guidewire 210 delivery of the working cannula 202, the interbody device 100, and the surgical instruments 212 may improve stability and precision throughout a procedure. The radiolucent portion 204 of the working cannula 202 may enable real-time visualization of the interbody device 100 at the surgical site during a procedure, thereby militating against injury and simplifying and optimizing the positioning and adjustment of the interbody device 100.

The interbody device 100, system 200, and method 300 may be advantageously configured for insertion of the interbody device 100 through the far lateral side of the disc space 123 using Kambin's triangle, thereby minimizing tissue, bone, nerve, and other damage while restoring a desired disc space height between the upper vertebra 125 and the lower vertebra 127, as well as improving lordosis. The use of Kambin's triangle as an access point may allow for a minimally invasive approach and improved surgical outcomes while bypassing the facet joint and militating against injury to bones, nerves, and tissues in and around the spine. Accordingly, the interbody device 100, system 200, and method 300 may reduce surgical complications and enhance surgical outcomes.

### EXAMPLE

### Minimally Invasive Lateral Lumbar Interbody Fusion Using Interbody Device

Example embodiments of the present technology are provided with reference to FIGS. 1-11.

A 45-year-old male patient may be experiencing chronic lower back pain and radiculopathy due to degenerative disc disease at the L4-L5 level. After alternative treatments do not provide adequate relief, the patient may elect to have a minimally invasive lateral lumbar interbody fusion procedure using the interbody device 100 described in this disclosure.

The procedure may begin with the patient positioned in a desired position on the operating table such that the disc space 123 may be accessed from the lateral side. Under fluoroscopic guidance, a small incision may be made on the patient's flank, targeting the L4-L5 disc space through Kambin's triangle. A guidewire 210 may be inserted through the incision and advanced to the target disc space 123, providing a reference for the subsequent steps of the procedure.

Following the placement of the guidewire 210, a series of cannulated dilators of increasing diameter may be sequentially inserted over the guidewire 210 to create a surgical pathway to the disc space 123. The gradual dilation technique may militate against tissue trauma while establishing a working channel of sufficient size to perform the procedure. Once the final dilator is in place, a working cannula 202 may be inserted over the series of dilators, and the series of dilators may be removed, leaving a clear path to the disc space 123 using the working cannula 202. The working cannula 202 may include a radiologically transparent or radiolucent portion 204 positioned adjacent the disc space 123.

Using surgical instruments 212 extending through the minimally invasive working cannula 202, a surgeon may perform discectomy and endplate preparation. A disc preparation instrument may be used to remove the degenerated disc material followed by a curette to clean the endplates of the adjacent vertebrae. An osteotome may be employed to create small channels in the vertebrae endplates, promoting blood flow and enhancing the fusion process.

Following preparation of the disc space 123, the interbody device 100 may be attached to an insertion instrument 214. The interbody device 100, measuring 10 mm in height with a 5-degree lordotic angulation measurement 126, may be selected based on preoperative imaging and intraoperative measurements. The insertion instrument 214 may then be used to guide the interbody device 100 through the working cannula 202 and into the prepared disc space 123. The interbody device 100 and the insertion instrument 214 may couple one another using one or more of a threaded connection, a sliding engagement mechanism, and/or a latch mechanism, as examples.

The radiolucent portion 204 of the working cannula 202 may allow the surgeon to visualize the interbody device 100 in real-time using fluoroscopy The shape of the insertion portion 116 of the interbody device 100 may facilitate easy, atraumatic entry into the disc space 123, while the convex top surface 124 may promote a desired lordosis correction. Once the interbody device 100 is properly positioned, the surgeon may use an expansion mechanism to increase or decrease a dimension or angulation measurement 126 of the interbody device 100 in order to optimally restore disc height and improve lordosis. The insertion instrument 214 may be decoupled from the interbody device 100 and carefully withdrawn from the surgical site using the working cannula 202.

Following placement of the interbody device 100, a bone graft material may be inserted through the central threaded hole 148 of the interbody device 100 using a bone graft delivery system. The bone graft material may fill the cavity 136 of the interbody device 100, thereby promoting fusion between the upper vertebra 125 and the lower vertebra 127. Fluoroscopic images may confirm the correct positioning of the interbody device 100 and the distribution of the bone graft material using the first opening 142, the second opening 144, and apertures 146 disposed in the interbody device 100 and/or radiolucent material 168 of the interbody device 100.

The minimally invasive approach, combined with the unique design features of the interbody device 100 and system 200 including the working cannula 202, guidewire 210, and surgical instruments 212, may result in optimal bone fusion and minimized tissue and nerve disruption. The patient may experience reduced postoperative pain compared to traditional open procedures and may be able to ambulate within hours of the surgery. Follow-up imaging at 3, 6, and 12 months post-surgery, as examples, may demonstrate optimal fusion and bone growth, maintenance of the restored disc height, and improved lordosis.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. An interbody device for a surgical procedure comprising:
an outer body made of a radiopaque material;
an insertion portion having a first width at a first end of the insertion portion that is less than a second width at a second end of the insertion portion, the insertion portion configured for entering into a disc space; and
a top surface having a convex shape.

2. The interbody device of claim 1, wherein the interbody device is configured for insertion at a far lateral side of the disc space through Kambin's triangle.

3. The interbody device of claim 1, wherein the insertion portion includes a substantially cone-like shape.

4. The interbody device of claim 1, wherein the insertion portion includes a substantially cylindrical shape at the insertion portion first end and includes a concave side wall surface extending from the insertion portion first end to the insertion portion second end.

5. The interbody device of claim 1, wherein the top surface includes an angulation measurement from greater than 0 degrees to 20 degrees.

6. The interbody device of claim 1, wherein the interbody device includes a plurality of apertures disposed in at least one of the first side wall and the second side wall.

7. The interbody device of claim 1, wherein the rear wall includes a threaded hole and a lateral slot configured to removably couple to the insertion instrument.

8. The interbody device of claim 1, wherein the interbody device includes a cannulation channel configured for guided insertion over a guidewire.

9. The interbody device of claim 1, wherein the interbody device includes an inner core made of a radiolucent material.

10. The interbody device of claim 9, wherein the interbody device includes the outer body made of a radiopaque material, the insertion portion having a first width at a first end of the insertion portion that is less than a second width at a second end of the insertion portion, the top surface having a convex shape, and the inner core made of a radiolucent material, and wherein the inner core includes at least one of an inner core opening disposed in at least one of a top wall and a bottom wall of the inner core and an inner core aperture disposed in a side wall of the inner core.

11. A system for a surgical procedure, comprising:
an interbody device having
an outer body made of a radiopaque material,
an insertion portion having a first width at a first end of the insertion portion that is less than a second width at a second end of the insertion portion, the insertion portion configured for entering into a disc space, and
a top surface having a convex shape; and
a working cannula, wherein the working cannula includes a radiolucent portion.

12. The system of claim 11, wherein a first end of the working cannula positioned adjacent a disc space is made of a radiolucent material.

13. The system of claim 11, wherein the system includes a guidewire configured to extend through the working cannula.

14. The system of claim 11, wherein the system includes a cannulated surgical instrument configured for guiding over the guidewire.

15. The system of claim 11, wherein the working cannula includes a series of dilators.

16. The system of claim 13, wherein the system includes a cannulated insertion instrument including the interbody device positioned over the guidewire.

17. A method of performing a procedure, the method comprising steps of:
providing an interbody device having
an outer body made of a radiopaque material,
an insertion portion having a first width at a first end of the insertion portion that is less than a second width at a second end of the insertion portion, the insertion portion configured for entering into a disc space, and
a top surface having a convex shape;
creating a surgical pathway to the disc space through Kambin's triangle;
inserting the interbody device through the surgical pathway and into the disc space using an insertion instrument removably coupled to the interbody device;
decoupling the insertion instrument from the interbody device; and
allowing fusion to occur between adjacent vertebrae.

18. The method of claim 17, wherein the method includes a step of inserting a guidewire to form a surgical pathway leading to a surgical site and a step of inserting a working cannula over the guidewire to the surgical site, the working cannula having a radiolucent portion disposed adjacent the surgical site.

19. The method of claim 17, wherein the method includes a step of inserting the insertion instrument including the interbody device over the guidewire to the surgical site.

20. The interbody device of claim 17, wherein the method includes a step of obtaining a radiographic image during the procedure to determine a position of the interbody device.
